(19) United States
(12) Patent Application Publication
(10) Pub. No.: US 2008/0288004 A1
(43) Pub. Date: Nov. 20, 2008

(54) TISSUE SUSPENSION DEVICE

(75) Inventor: Stephen A. Schendel, Menlo Park, CA (US)

Correspondence Address: CHALKER FLORES, LLP, 2711 LBJ FRWY, Suite 1036, DALLAS, TX, 75234, US

(73) Assignee: GENESIS BIOSYSTEMS CORPORATION, Lewisville, TX (US)

(21) Appl. No.: 12/122,512

(22) Filed: May 16, 2008

Related U.S. Application Data
(60) Provisional application No. 60/938,383, filed on May 16, 2007.

(51) Int. Cl. A61B 17/68

(52) U.S. Cl. 606/331

(57) ABSTRACT

The present invention is an apparatus for suspending a soft tissue near a bone opening, which includes a bone anchor, a soft tissue anchor, a spacer, and a depth stop. The device of the present invention allows soft tissues to change positions and be held until sufficient healing has occurred.

### CROSS-REFERENCE TO RELATED APPLICATIONS

This application claims priority to U.S. provisional Application No. 60/938,383 filed on May 16, 2007. The entire contents of which are incorporated herein by reference.

### TECHNICAL FIELD OF THE INVENTION

The present invention relates in general to the field of soft tissue surgery, and more particularly, to devices, methods and systems for tissue suspension.

### BACKGROUND OF THE INVENTION

Without limiting the scope of the invention, its background is described in connection with soft tissue repair.

One such apparatus is shown in U.S. Pat. No. 5,441,502 filed by Bartlett. The '502 patent describes a system and method for re-attaching soft tissue to bone. The '502 application comprises a system with pin means, sleeve means, driver means, suture anchor means and suture anchor installation tool means. In use, a soft tissue is placed against the surface of the bone in its desired re-attachment position. Then the pin means is passed through the soft tissue and into a bone to securely fix the pin means (and hence the soft tissue) to the bone. Next, a patient's anatomy is manipulated as needed to determine if the soft tissue has been properly positioned relative to the bone. If the soft tissue is properly positioned, a surgeon proceeds to the next step in the procedure; if not, the pin means is removed and the tissue positioning operation repeated until the soft tissue has been positioned in the desired location. Next, the sleeve means is slid telescopically over the pin means, passed through the soft tissue and then, using the driver means, drilled into the bone. Then the pin means is completely withdrawn from the bone and the soft tissue, leaving a hole in the bone. The suture anchor means is then passed down the interior of the sleeve means and into the bone, using the suture anchor installation tool means. Then the installation tool means is withdrawn from the sleeve means, leaving the suture anchor means deployed in the bone and its associated suture extending out through the sleeve means. The sleeve means is then withdrawn, and the free ends of the suture used to secure the soft tissue to the bone.

Another example is shown in U.S. Pat. No. 5,634,926 by Jobe. The '926 patent is directed to a surgical bone fixation apparatus that teaches a bone fixation apparatus for anchoring a surgical member to bone for the fixation of bone or soft tissue to bone. The bone fixation apparatus includes a post device having a leg portion slidably insertable into a hole formed in bone. The leg portion is of sufficient length relative to the interior diameter of the hole to resist removal of the leg portion from the hole when forces substantially parallel to the outer surface of the bone are applied to the post device to anchor the surgical member to bone. In one aspect of the '926 patent, the surgical member is a plate section of an elongated sheet of surgical plate material which may be divided into a plurality of plate sections each having an arbitrary shape.

Yet another example is demonstrated in U.S. Pat. No. 6,015,410 by Tormala et al., for a bioabsorbable surgical implants for endoscopic soft tissue suspension procedure. The '410 patent teaches a bioabsorbable surgical implant for use in supporting soft tissue in a superior position in the body. The surgical implant includes a shaft that connects the implant to a bone or hard tissue and a head secured to the shaft. The head of the implant has a concave lower surface so that a suture (or sutures) can easily be wound around the shaft below the head and locked to this position by tightening the head against bone or hard tissue and by pushing or turning the shaft into the bone or hard tissue. The connected shaft and head are formed from a resorbable polymer, copolymer, polymer alloy or composite, which maintains a specified strength for a period of time at least equal to a healing period for the patient. Thereafter, the surgical implant is substantially resorbed by the body over a period of time needed for healing. The surgical implant is particularly adapted for use in endoscopic face and/or brow lift surgery and other endoscopic cosmetic, plastic and reconstructive surgical procedures, where sutures are applied for tissue lifting.

Yet another example is demonstrated in U.S. Pat. No. 6,866,666, issued to Sinnott, et al., which discloses a system and method for attaching soft tissue to bone. The system and method for attaching soft tissue to bone includes a novel two-part anchor for attaching soft tissue and the like to bone. The two-part anchor generally includes a stake and a cap. The stake taught by Sinnott is adapted to be positioned in bone and form a stake for impalement by a piece of soft tissue. The cap is adapted to cap soft tissue that has been impaled on the stake and thereby bind the soft tissue to the stake and, hence, to the bone in which the stake is set.

Another system is shown in U.S. Pat. No. 7,156,862 by Jacobs for a multi-point tension distribution system device and method for tissue approximation using the device to improve wound healing. The '862 patent taught an implantable, biodegradable construct (except for hernia repairs) that has multiple attachment points emanating from a supportive backing. The device improves the mechanical phase of wound healing and evenly distributes tension over the contact area between the device and tissue. Processes for using the device include wound closure, vascular anastomoses, soft tissue attachment and soft tissue to bone attachment.

Yet another system is shown in United States Patent Application Publication No. 20050197699 by Jacobs for a tissue repair apparatus and method. The '699 application teaches the use of one or more tine plates for repairing tissue (e.g., hand/wrist tendons). Each plate has a center portion includes multiple fenestrations, and/or a width or thickness that is less than that of first and second end portions of the plate, The center portion is more pliable than the first and second end portions. A first group of tines extends from the first end portion non-orthogonally and angled toward the center portion, and a second plurality of tines extends from the second end portion non-orthogonally and angled toward the center portion. Finally, a suture, button or ring is used to affix the plate(s) to the tendon. The plate(s) extends across a sever point to serve as the load-bearing member, wherein the tines are positioned on both sides of the tendon sever point to fix the tissue.

Finally, United States Patent Application Publication No. 20050209542 by Jacobs et al. demonstrates a tissue approximation sling and method. The '542 application is said to teach a medical sling device having a pair of tissue approximating devices each including a supportive backing member, a plurality of tissue engagement tines extending from the backing member, and an elongated extension member extending from the backing member. Each extension member is slidably engaged with the back member of the other tissue approximating device, such that pulling on the first and second extension members causes the first and second backing members to slide toward each other.

### SUMMARY OF THE INVENTION

Currently, a new subcutaneous medical device is in need for surgeons to improve the healing time and ease of installation. The present invention is geared towards that need. Various embodiments of the subcutaneous medical device and methods of making and installation are described herein.

The present inventors recognized that during surgery, it is frequently necessary to suspend soft tissues in order to change there position. This type of position changes are frequently needed in cosmetic and reconstructive surgery, such as in the area of the brow, eyes and face. The soft tissues typically must be held in a new position until sufficient healing has occurred; failure to do this causes the tissues to move back towards their original position thus undoing the desired results.

The present invention is a subcutaneous medical device including a biodegradable, soft tissue peg having a first portion adapted to insert into a bone and a second portion, opposite the first portion adapted to retain soft-tissue. In addition, the subcutaneous medical device may have a spacer and/or depth stop between the first portion and the second portion. The peg may be made with biodegradable or biocompatible materials such as poly(ethylene-co-vinyl acetate), poly(DL-lactide), poly(glycolide), copolymers of lactide and glycolide, and polyanhydride. The first portion, second portion, spacer, and the depth stop of the present invention may be made out of different materials, and may include active agents or coating of active agents such as anti-inflammatory, analgesic, local anesthetic, and combinations thereof.

The present invention includes the first portion of the subcutaneous medical device, called a bone anchor, the second portion called the soft tissue anchor, a spacer, and optionally a depth stop for depth control and/or mechanical support. Generally, the bone anchor is adapted to insert into a bone opening or orifice; the bone opening, as well as the bone anchor, may exhibit different shapes, sizes, length, and/or depth. The bone anchor typically has a total perimeter measurement smaller than the bone opening to provide a unique fit. The device may also include a self-locking mechanism. The bone anchor may be positioned on different parts of skeletal structure depending on surgical needs.

The soft tissue anchor of the present invention is adapted to retain a soft tissue such as periosteum, muscles, fascia, and skin to the underlying skeleton (e.g., during facial reconstruction); it may also exhibit different shapes, sizes, and thickness. For example, the soft tissue anchor may take the shapes of a spike, a hook, or even one half of a stem and loop structure, depending on the type of soft tissue needed to be place. The soft tissue anchor of the present invention may be varied in length, thickness and shape to accommodate placement in different locations depending on the type and visibility of the soft tissue. For example, different portions of the face may require the use of soft tissue anchors with different lengths to minimize visibility while at the same time providing the required mechanical integrity to allow for repair of the soft tissue.

The present invention also includes a spacer and/or depth stop between the bone anchor and the soft tissue anchor. The spacer can ideally take on different shapes, sizes, and/or positions on the subcutaneous medical device to provide various measurable insertion depth and insertion angle into an opening of a bone.

In one embodiment of the present invention, the bone anchor and soft tissue anchor are pointing away from one another connected by a linear spacer. The soft tissue anchor is angled against the spacer, and the bone anchor is angled against the same spacer in an opposite direction, resulting in a continuous “Z” shaped subcutaneous medical device.

Yet another embodiment of the present invention is a peg with branched spacer resulting in a “V” shape subcutaneous medical device with a soft tissue anchor at the branched both extremities, and a bone anchor at the base of the “V”.

The tissue suspension device taught herein generally may be used by making one or more openings in a bone adjacent a soft tissue; placing one or more subcutaneous pegs having the bone anchor adapted to insert into the bone openings, and the soft tissue anchor adapted to retain soft-tissue. Next, soft tissue is stretched over the soft tissue anchor of the peg, and engaged to the soft tissue anchor. A user can control the exact location, stress and pressure points of the soft tissue on the peg during soft tissue reconstruction. Variations of the subcutaneous medical device and methods of installation are described herein. Also described is a tool called the bone anchor positioning guide that is particularly useful for create bone openings.

### BRIEF DESCRIPTION OF THE DRAWINGS

For a more complete understanding of the features and advantages of the present invention, reference is now made to the detailed description of the invention along with the accompanying figures and in which:

FIG. 1 illustrates a subcutaneous medical device.

FIGS. 2A-2B illustrates multiple embodiments of a subcutaneous medical device.

FIG. 3 illustrates a continuous “Z” shaped subcutaneous medical device.

FIG. 4 illustrates a subcutaneous medical device with a branched spacer.

FIG. 5 illustrates one orientation of the bone opening.

FIGS. 6A-6B illustrates the bone anchor positioning guide top and side view.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Moving tissue presents unique challenges, as tissues often resist closure, depending on the nature of the tissue structure, the circumstances of the tissue separation, and general patient health. Complications related to wound closure and healing generally result from forces or compromised healing responses. These forces are retractive forces created beyond the viscoelastic properties of the tissue, and may be created by: increased internal volume, such as in the case of obesity, which elevates containment forces on the skin system; changes in wound aspect ratio, such as increased abdominal circumference created in a prone, non-ambulatory patients due to muscular atrophy; respiratory muscular activity; muscular response; loss of fascia structure; muscular-skeletal deformation; fleshy appendages; tumors; and severe burns.

Other forces such as minor forces are internal forces created by the viscoelastic properties of the tissue, which can cause the skin to retract. Elastic tissues, such as skin, return to a minimum elastic state when released from tension. In this relaxed state, tissue cells have a spherical shape, cell walls are thick and strong, and cell surface tensions are minimized and balanced. A cell in this relaxed state will remain stable, behaving similarly to a non-elastic material. The force required to elongate a cell in this state often approaches a force that will rupture or sheer intercellular bonds, causing localized failures or tears. Soft tissue in this minimum elastic state provides minimum surface coverage and has the highest reluctance to stretch. It is believed that a gentle but constant force below the sheer force threshold applied to tissue in combination with adequate hydration will, over time, restore certain tissues to original elastic state. Additionally, this force can be applied to stretch tissue past the point of equilibrium to the maximum elastic range and create the thinnest possible configuration, covering the maximum surface area. If intercellular pressures in the tissue do not exceed the point at which intercellular bonds are compromised, the tissue remains at the maximum elastic state as healthy tissue, and normal biological processes will build additional cells to restore normal skin thickness and tension.

In addition, compromised healing responses may complicate wound closure or healing. An incision becomes a wound as soon as it falls behind normal healing protocol. Wound management, including treatment and care of large skin defects and severely retracted incisions, is an area of increasing importance to the health care community. An aging population and an increase in diseases related to obesity and inactivity have increased the occurrence of chronic wounds and place an increased burden on health care resources. Factors contributing to compromised wound healing include patient age, weight, nutritional status, dehydration, blood supply to the wound site, immune response, allergies to closure materials, chronic disease, debilitating injuries, localized or systemic infection, diabetes, and the use of immunosuppressive, corticosteroid or antineoplastic drugs, hormones, or radiation therapy. Chronic wounds include, but are not limited to: diabetic ulcers and other chronic ulcers; venous stastis ulcers; pressure sores or ulcers; burns; post traumatic lesions, such as post disarticulation, post debridement, cutaneous gangrene, post colectomy, crush wounds with ischemic necrosis; collagen disease, including rheumatoid arthritis; vasculitis (lesions and ulcers caused by arterial insufficiency); amputation; fasciotomy; post surgical dehiscence; post sternotomy; necrotising fasciitis; trauma; wounds having exposed plates or bones; scar revision; skin lesions; blunt abdominal trauma with perforations; pancreatitis; neuropathic ulcers; compartment syndrome; and other subacute or chronic wounds. Treatment and care of these defects is challenging due to difficulties in closure of open wounds.

Two common methods of closure of wounds and skin defects include split thickness skin grafting and gradual closure. A split thickness skin graft involves removing a partial layer of skin from a donor site, usually an upper leg or thigh, and leaving the dermis at the donor site to re-epithelialize. In this manner, a viable skin repair patch can be transferred or grafted to cover a wound area. The graft is often meshed, (which involves cutting the skin in a series of rows of offset longitudinal interdigitating cuts) allowing the graft to stretch to cover two or three times greater an area as well as provide wound drainage while healing. Normal biological function of the skin heals the holes after the graft has been accepted. A meshed graft of this type requires a smaller donor area than a conventional non-meshed or full thickness skin graft. However, these methods do not provide optimal cosmesis or quality of skin cover. Other disadvantages of this method include pain at the donor site, creation of an additional disfiguring wound, and complications associated with incomplete “take” of the graft. In addition, skin grafting often requires immobilization of the limb, which increases the likelihood of contractures. The additional operation and prolongation of hospital stay is an additional economic burden.

The present inventors recognized that during surgery, it is frequently necessary to suspend soft tissues in order to change there position. This type of position changes are frequently needed in cosmetic and reconstructive surgery, such as in the area of the brow, eyes and face. The soft tissues typically must be held in a new position until sufficient healing has occurred; failure to do this causes the tissues to move back towards their original position thus undoing the desired results. It is particularly challenging to adhere soft tissues to the bone, in particular, adhering periosteum, muscles, fascia and skin of the face to the underlying skeleton. Sutures alone have proven inadequate to maintain the desired soft tissue result and multiple apparatus have been proposed to aid in this soft tissue suspension.

The present invention is a subcutaneous medical device that can overcome the challenges described. Various embodiments of the subcutaneous medical device and methods of making and installation are described herein.

The present invention is a subcutaneous medical device in the form of a peg that is positioned into an opening in a bone and permits an overlying of soft tissues in a controlled manner in order to adhere to one or more sharp ends of the peg to the soft tissue. Facial areas where the subcutaneous medical device is frequently used, but not limited to, are the forehead (e. g., in a brow lift), the midface (e. g., in a midface lift), the eyes (e. g., after eyelid surgery), and the chin. The peg may also be used to attach periosteum and/or muscles to the underlying bone as is frequently done in craniofacial surgery where the temporalis muscle is detached during the surgery. Other examples of soft tissues include cartilage, tendon or ligament.

The subcutaneous medical device of the present invention may be made from any biocompatible material. The material may also be biodegradable. Biocompatible materials include, but are not limited to, titanium, stainless steel, ceramic, glass, wood, cellulose, and/or combinations thereof. In addition, the subcutaneous medical device may be made out of biodegradable natural or synthetic polymer such as poly(ethylene-co-vinyl acetate), poly(DL-lactide), poly(glycolide), copolymers of lactide and glycolide, and polyanhydride copolymers and/or combinations thereof. The subcutaneous medical device of the present invention may be sterilized using any of the well known techniques, depending on the material used in manufacturing of the device. In addition, the subcutaneous medical device can be artificially colored to provide camouflage effects to the surrounding soft tissues.

Generally, the biodegradable and/or biocompatible material will exhibit a tensile strength that provides structural integrity to anchor the bone and the soft tissue. The material may also include various active agents, and/or coating of various active agents. The active agents that may be added or coated to the device include a variety of pharmaceutical compositions and bone-inducing compositions, as well as biologics and bio-active materials. The active agents include, but are not limited to, anti-inflammatory agents, analgesic agents, local anesthetic agents, and combinations thereof. In one embodiment, the peg may also comprise a core or pin that increases the mechanical strength of the peg. The pin may even be non-biodegradable and can be allowed to remain or easily be removed after the soft tissue heals through a single, small orifice. If the pin is metal, it can easily be identified with a metal detection device. In some embodiments, the pin is positioned such that it is able to remain after a biodegradable peg dissolves and has a length that traverses the periosteum (to maintain mechanical strength of attachment) but does not traverse the soft tissue.

Examples of anti-inflammatory agents include, but are not limited to, algestone, amicinonide, beclomethasone, cortisone, difluprednate, enoxolone, formocortal, halcinonide, hydrocortisone, loteprednol etabonate, mometasone furoate, oxametacine, prednisolone, prednisolone 25-diethylaminoacetate, rimexolone, triamcinolone, xenbucin, and pharmaceutically acceptable salts thereof.

Examples of analgesic agents include, but are not limited to, acetaminophen, anileridine, benzylmorphine, bezitramide, codeine, choline salicylate, dimenoxadol, dimepheptanol, dimethylthiambutene, dioxaphetyl butyrate, etonitazene, fentanyl, hydrocodone, isomethadone, ketobemidone, levorphanol, meperidine, narceine, opium, piminodine, rumifentanil, tilidine, and pharmaceutically acceptable salts thereof

Examples of local anesthetic agents include, but are not limited to, ambucaine, amolanone, butethamine, cyclomethycaine, dibucaine hydrochloride, ecgonine, fomocaine, hexylcaine hydrochloride, hydroxytetracaine, isobutyl p-aminobenzoate, leucinocaine mesylate, myrtecaine, naepaine, octacaine, phenol, ropavacaine, salicyl alcohol, tetracaine hydrochloride, tolycaine, zolamine, and pharmaceutically acceptable salts thereof.

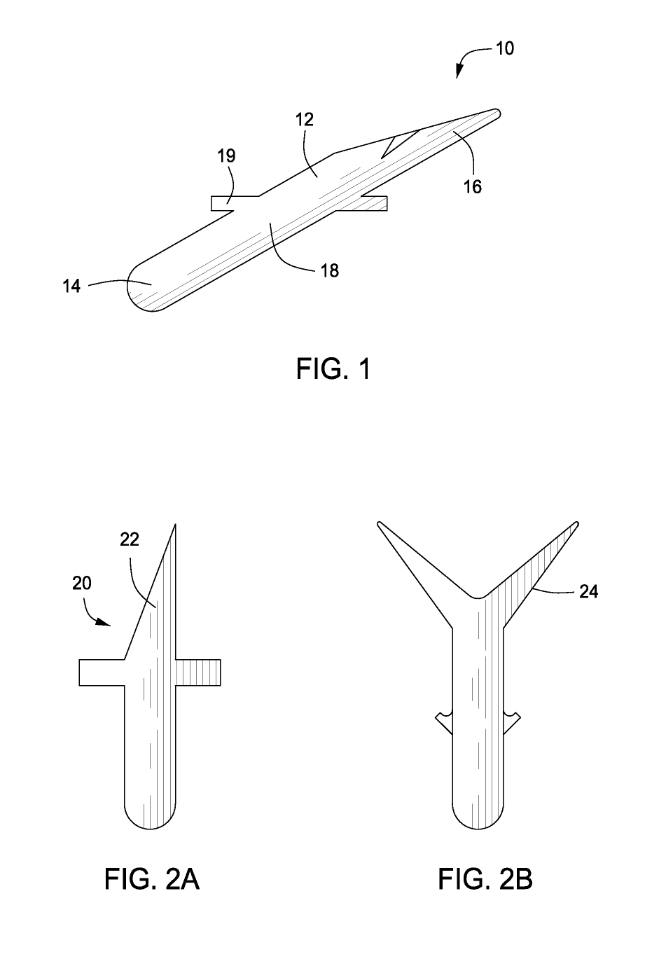

FIG. 1 illustrates a subcutaneous medical device 10 that includes a single, continuous peg 12 with a first portion bone anchor 14 to be inserted into an opening in a bone, and a second portion soft tissue anchor 16 to retain soft tissue. The two portions 14 and 16 are opposite to each other and are shown with a spacer 18 there between. A spacer 18 can ideally take on different shapes, sizes, and/or positions on the peg to provide various measurable distance between the bone and the soft tissue anchor. The present invention also includes a depth stop 19, typically but no necessarily located in the spacer region. The depth stop may also take on different materials, shapes, sizes, and thickness, and act as a guide to control the exact insertion depth into the bone opening. The spacer 18 may also be used in conjunction with a delivering device (not shown in this figure) that the surgeon uses to deliver and position the device 1O.

The bone anchor 14 shown in FIG. 1 is typically blunt ended, but may take on different shapes, sizes, thickness, and length depending on the size and shape of the bone opening. The soft tissue anchor 16 is typically sharp-ended in order to hold a soft tissue. Soft tissue anchor 16 can take on multiple shapes and/or sizes or be branched as illustrated in FIGS. 2A and 2B. FIG. 2A illustrates one embodiment of a subcutaneous medical device 20 with a cone shaped soft tissue anchor 22. In another embodiment, shown in FIG. 2B, the soft tissue anchor 24 is multi-branched with arrows pointed away from each other and in various, different directions.

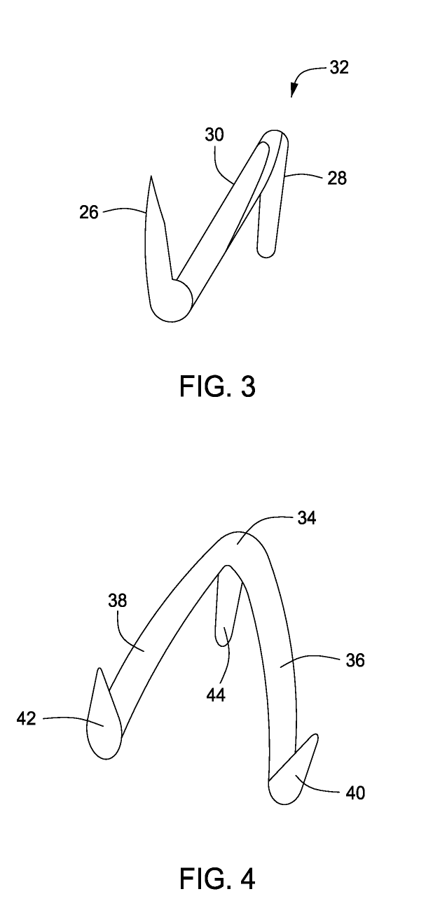

In addition, the device 10 can take on other geometric shapes other than linear. In one preferred embodiment shown in FIG. 3, bone anchor 26 and soft tissue anchor 28 are pointing away from one another connected by a linear spacer 30. The soft tissue anchor 26 is angled against spacer 30, and the bone anchor 28 is angled against spacer 30 in the opposite direction, resulting in a continuous “Z” shaped subcutaneous medical device 32. The spacer 30 can vary in length, shape, thickness, or material; therefore, provides flexibility for a surgeon to decide the placement of the peg and vary the distance between 26 and 28. In another embodiment, shown in FIG. 4, the peg 34 has branched spacer 36 and 38 resulting in a “V” shape with soft tissue anchor 40 and 42 at the branched extremities, and bone anchor 44 at the base of the “V”. The embodiment depicted in FIG. 4 provides additional anchoring force to hold the overlaying soft tissue to the bone anchor 26. Although this paragraph descried certain embodiments including geometric shapes of “Z” and “V”, a skilled artisan would recognize that a variety of shapes can also be used for the present invention.

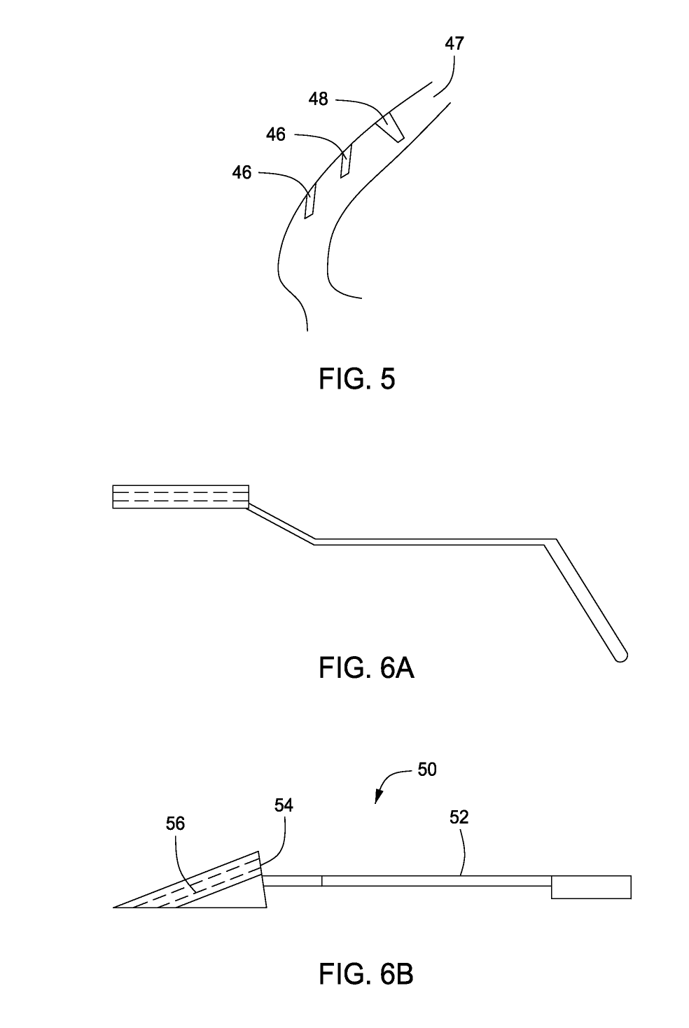

FIG. 5 illustrates one orientation of a bone opening 46 relative to the bone segment 48. It is contemplated that the bone opening 46 may be positioned in different part of the human skeleton system including, but not limited to, skull, femur, tibia, gibula, humerus, radius, ulna, metacarpals, phalanges, metatarsals, clavicle, innominate, scapula, ribs, sternum, carpal, tarsal, and patella bone. In addition, the bone opening may be made in different types of bone such as compact bone and cancellous bone. In the non-limiting embodiment described, the subcutaneous medical device is positioned on facial and cranial bone.

The bone opening in the bone can take different shapes, sizes, and depth depending on the need of the surgical operation. Shapes for the bone opening may be, but not limited to, circular, semicircular, triangular, rectangular, hexagonal, pentagonal, octagonal, and heptagonal. In FIG. 5, the bone opening 46 is oblique, monocortical, and is angled approximately thirty degrees against the surface of the bone segment 47. Generally, the bone opening will be created using a drilled and drill bit. The slanted or angled bone opening 46 permits a user with a tool to come in from a distance to create the bone opening at an ideal level or position. In addition, the bone opening may have one or more slanted walls, as shown in 48; thereby providing a unique fit to the bone anchor.

FIG. 6A denotes a top view, and FIG. 6B denotes a side view of the bone anchor positioning guild. The bone opening 46 may be drilled, ground carved, or punched using a bone anchor positioning guide 50 shown in FIG. 6. The bone anchor positioning guide includes a handle portion 52 and a guidance portion 54. The guidance portion 54 is internally modified with a tunnel opening 56 to guide a user's tool such as a drill to produce a slanted or angled opening into a bone. The handle portion 52 is angled away from the tunnel 56 to allow clear access of a drill bit without obstructing the drilling action or a user's view. The slanted or angled monocortical bone opening 46 may be drilled with a diameter slightly larger than that of the bone anchor. The bone anchor may be screwed, glued, and/or pressure fitted into the bone opening. A self-locking mechanism may also be provided. Next, a soft tissue can be positioned over the peg that will support the soft tissue in the new position. One or more pegs may be placed independently until the surgeon feels there is sufficient retention of the soft tissue. The density, distribution, and orientation of soft tissue anchors may be varied depending on surgeon's need to reduce damages to the soft tissue.

In certain embodiments, the present invention can be made out of porous materials. For soft tissue applications, the properties of porous materials have an important role. Similar to bone, the overwhelming volumetric porosity allows fast penetration of precursor cells and relatively fast formation of soft tissue fibril strands and blood supply. Unlike solid metal screws, washers or synthetic sutures, porous material of the present invention achieves the primary mode of tissue attachment to the implant device while the tissues heal at their own variable pace. The struts of the porous material interlock can with the tissue, offering immediate, secure and functional mechanical attachment. This allows for the necessary healing and reproducible tissue incorporation into the porous matrix. The use of a porous tissue anchoring device may therefore result in both soft tissue in-growth and bone in-growth for long-term fixation.

In certain embodiments, the present invention is more adaptable in that one or more tissue tacs can be applied in differing arrangements (e.g., positions and orientations), thus the present invention is more readily to be modified depending on the patients specific anatomy and needs. Most of the existing device cited in prior references comes in set shapes with multiple hooks and are not modifiable. As such, the present invention teaches away from the prior references and solves a long felt need for patients. In addition, the present inventors recognize that the amount of resorbable material of some prior reference also causes medical and health concerns due to their large size and material characteristics. The present inventor also recognized that such large device can be palpable at times and sterile abscecess have been reported as the large amount of material is resorbed. Furthermore, prior references teaches fixation of their large device far removed from the actual hook and can not be rearranged closer without making another unsightly incision because of device configuration. In contrary, the present invention can be applied using a remote incision (e.g., endoscopically) and the fixation point to the underlying bone is the same. Accordingly, the present invention differentiates from the prior references and provides stable results, better esthetics with fewer complications.

In another embodiment, the present invention differentiates from prior reference by provides significantly miniaturized device, thus reduces intrusion to patients and provide unexpected, surprising speed of healing. In one embodiment, the present invention has a “Z” shape and has a dimension of 1 mm length by 6 mm width. This ultra-miniaturized device can be place by any remote incision surgical techniques without unnecessary incisions.

It should be appreciated that the present invention can be made by other open cell materials, such as high temperature ceramics. In addition, other layers may be deposited on the substrate, such as intermediate layers to provide additional strength. Other aspects of the invention could be the incorporation of a core of solid material, such as tantalum or niobium or alloys of each, with the porous substrate fitted around the solid core and with the subsequent deposition of metal not only covering the substrate but also locking the porous substrate to the solid core.

The device described herein may be used as part of any appropriate procedure. As mentioned above, the treatments listed are only one example of a procedure that may benefit from the anchors described herein. In general, the flexible tissue anchors described herein may be used to connect tissue to tissue or an implant or graft to a tissue, or a graft to a graft, or to form an anchoring system for reshaping tissue.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A subcutaneous medical device comprising:
a biodegradable, soft tissue peg having a first portion adapted to insert into a bone and a second portion, opposite the first portion adapted to retain soft-tissue.

2. The device of claim 1, wherein the peg comprises at least one of poly(ethylene-co-vinyl acetate), poly(DL-lactide), poly(glycolide), copolymers of lactide and glycolide, and polyanhydride copolymers.

3. The device of claim 1, wherein at least a portion of said subcutaneous medical device comprises one or more active agents to facilitate healing.

4. The device of claim 1, wherein the soft tissue comprises periosteum, muscles, fascia and skin of the face and the portion adapted to retain soft-tissue.

5. The device of claim 1, further comprising a spacer between the first portion and the second portion.

6. The device of claim 1, further comprising a depth stop between the first and second portion of the peg.

7. The device of claim 1, wherein the peg is self-locking.

8. The device of claim 1, wherein the second portion is adapted to retain the soft tissue comprises a spike, a claw, or one half of a stem and loop structure.

9. The device of claim 1, wherein a second half of a stem and loop structure is positioned on the soft tissue.

10. The device of claim 1, wherein the first and second portions are made from different materials.

11. A subcutaneous medical device comprising:
a self-locking, soft tissue peg having a first portion adapted to insert into a bone and a second portion, opposite the first portion adapted to retain soft-tissue.

12. The device of claim 11, wherein the peg comprises at least one of poly(ethylene-co-vinyl acetate), poly(DL-lactide), poly(glycolide), copolymers of lactide and glycolide, or polyanhydride copolymers.

13. The device of claim 11, wherein the peg comprises at least one of stainless steel, titanium, chrome, cellulose, ceramic, glass or plastic.

14. The device of claim 11, wherein the soft tissue comprises periosteum, muscles, fascia and skin of the face and the portion adapted to retain soft-tissue.

15. The device of claim 11, wherein at least a portion of said subcutaneous medical device comprises one or more active agents to facilitate healing.

16. The device of claim 11, further comprising a spacer between the first portion and the second portion.

17. The device of claim 11, further comprising a depth stop between the first and second portion of the peg.

18. The device of claim 11, wherein the peg is biodegradable.

19. The device of claim 11, wherein the second portion is adapted to retain the soft tissue comprises a spike, a hook, a claw or one half of a stem and loop structure.

20. The device of claim 11, wherein a second half of a stem and loop structure is positioned on the soft tissue.

21. A method of retaining soft tissue comprising the steps of:
making one or more orifices in a bone adjacent a soft tissue;
aligning one or more subcutaneous medical device comprises a bone anchor and a tissue anchor over said orifice;
fitting said bone anchor into said orifice;
positioning said soft tissue over said subcutaneous medical device; and
retaining said soft tissue using said soft tissue anchor.

22. The method of claim 21, wherein at least a portion of said subcutaneous medical device comprises biocompatible material.

23. The method of claim 21, wherein at least a portion of the anchors said subcutaneous medical device comprises biodegradable material.

24. The method of claim 21, wherein at least a portion of the said subcutaneous medical device comprise one or more active agents that can facilitate healing.

25. The method of claim 21, further comprising a self-locking mechanism to fix said subcutaneous medical device in the deployed configuration.

26. A subcutaneous medical device comprising:
an angled, biodegradable, soft tissue peg having a first portion adapted to insert into a bone at an angle and a second portion, opposite the first portion adapted to retain soft-tissue.

27. The device of claim 26, wherein the peg comprises at least one of poly(ethylene-co-vinyl acetate), poly(DL-lactide), poly(glycolide), copolymers of lactide and glycolide, and polyanhydride copolymers.

28. The device of claim 26, wherein at least a portion of said subcutaneous medical device comprises one or more active agents to facilitate healing.

29. The device of claim 26, wherein the soft tissue comprises periosteum, muscles, fascia and skin of the face and the portion adapted to retain soft-tissue.

30. The device of claim 26, further comprising a spacer between the first portion and the second portion.

31. The device of claim 26, further comprising a depth stop between the first and second portion of the peg.

32. The device of claim 26, wherein the peg further comprises a pin coaxial with the peg.

33. The device of claim 26, wherein the second portion is adapted to retain the soft tissue comprises a spike, a hook, a claw or one half of a stem and loop structure.

34. The device of claim 26, wherein a second half of a stem and loop structure is positioned on the soft tissue.

35. The device of claim 26, wherein the first and second portions are made from different materials.
